(19) United States
(12) Patent Application Publication
(10) Pub. No.: US 2024/0122883 A1
(43) Pub. Date: Apr. 18, 2024

(54) METHODS OF TREATING A SUBJECT EXPOSED TO A TOXIC INHALED CHEMICAL WITH MESNA

(71) Applicants: NATIONAL JEWISH HEALTH, Denver, CO (US); SOUTH DAKOTA STATE UNIVERSITY, Brookings, SD (US); THE REGENTS OF THE UNIVERSITY OF COLORADO, A BODY CORPORATE, Denver, CO (US)

(72) Inventors: Carl W. WHITE, Denver, CO (US); Heidi NICK, Arvada, CO (US); Preston BRATCHER, Arvada, CO (US); Brian LOGUE, Brookings, SD (US)

(21) Appl. No.: 18/264,200

(22) PCT Filed: Feb. 15, 2022
(86) PCT No.: PCT/US2022/016445

§ 371 Date: Aug. 3, 2023

Related U.S. Application Data
(60) Provisional application No. 63/149,973, filed on Feb. 16, 2021.
(60) Provisional application No. 63/186,565, filed on May 10, 2021.

(51) Int. Cl. A61K 31/185; A61K 38/49; A61P 39/02

(52) U.S. Cl. CPC A61K 31/185; A61K 38/49; A61P 39/02

(57) ABSTRACT

In one aspect, the present disclosure provides a method of treating a subject exposed to as toxic inhaled chemical, the method comprising administering to the subject a therapeutically effective amount of 2-mercaptoethane sulfonic acid, or a salt or solvate thereof. In some embodiments, the method further comprises administering to the subject a therapeutically effective amount of tissue plasminogen activator. In some embodiments, the toxic inhaled chemical is sulfur mustard, methyl isocyanate, or a combination thereof.

### CROSS-REFERENCE TO RELATED APPLICATIONS

The present application claims priority under 35 U.S.C. § 119(e) to U.S. Provisional Patent Applications No. 63/149,973, filed Feb. 16, 2021, and No. 63/186,565, filed May 10, 2021, all of which are incorporated herein by reference in their entireties.

### STATEMENT REGARDING FEDERALLY SPONSORED RESEARCH

This invention was made with government support under contract number 5U54ES027698 awarded by National Institutes of Health. The government has certain rights in the invention.

### BACKGROUND

Human exposure to toxic inhaled chemicals can occur due to warfare, terrorism, and/or industrial and transportation accidents. The current pandemic demonstrates the need for vigilance to chemical, biological, radiation, and nuclear (CBRN) threats to our society. There are currently no FDA-approved therapies for Toxic Inhaled Chemicals (TICs).

There is a need in the art for a method of treating a subject who has been exposed to a TIC. The present invention satisfies this unmet need.

### BRIEF SUMMARY OF THE INVENTION

In one aspect, the invention provides a method of treating a subject exposed to a toxic inhaled chemical, the method comprising administering to the subject a therapeutically effective amount of 2-mercaptoethane sulfonic acid, or a salt or solvate thereof.

In certain embodiments, the 2-mercaptoethane sulfonic acid salt is sodium 2-mercaptoethane sulfonate or a solvate thereof.

In certain embodiments, the toxic inhaled chemical is sulfur mustard, chlorine gas, methyl mercaptan, nitrogen mustard, 2-chloro-ethyl-ethylsulfide (CEES), methyl isocyanate, or a combination thereof.

In certain embodiments, 2-mercaptoethane sulfonic acid, or salt or solvate thereof, is administered to the subject via one of the following routes: oral, intravenous, subcutaneous, intra-osseus, intramuscular, intraperitoneal, intratracheal, cutaneous, or intra-ocular.

In certain embodiments, between about 120 mg/m 2 to about 720 mg/m 2 of 2-mercaptoethane sulfonic acid, or a salt or solvate thereof, is administered to the subject.

In certain embodiments, about 120 mg/m 2 to about 360 mg/m 2 of 2-mercaptoethane sulfonic acid, or a salt or solvate thereof, is administered intravenously to the subject.

In certain embodiments, about 240 mg/m 2 to about 720 mg/m 2 of 2-mercaptoethane sulfonic acid, or a salt or solvate thereof, is administered orally to the subject.

In certain embodiments, 2-mercaptoethane sulfonic acid, or a salt or solvate thereof, is administered to the subject immediately to about ten hours after the subject was exposed to the toxic inhaled chemical.

In certain embodiments, 2-mercaptoethane sulfonic acid, or a salt or solvate thereof, is administered to the subject within about 20 minutes after the subject was exposed to the toxic inhaled chemical.

In certain embodiments, 2-mercaptoethane sulfonic acid, or a salt or solvate thereof, is administered to the subject about two hours to about eight hours after the subject was exposed to the toxic inhaled chemical.

In certain embodiments (i) a first dose of 2-mercaptoethane sulfonic acid, or a salt or solvate thereof, is administered orally and/or intravenously to the subject within about 20 minutes after the subject was exposed to the toxic inhaled chemical; (ii) a second dose of 2-mercaptoethane sulfonic acid, or a salt or solvate thereof, is administered orally and/or intravenously to the subject about 4 hours after the subject was exposed to the toxic inhaled chemical; and (iii) a third dose of 2-mercaptoethane sulfonic acid, or a salt or solvate thereof, is administered orally and/or intravenously to the subject about 8 hours after the subject was exposed to the toxic inhaled chemical.

In certain embodiments, (i) a first dose of 2-mercaptoethane sulfonic acid, or a salt or solvate thereof, is administered intravenously to the subject within about 20 minutes after the subject was exposed to the toxic inhaled chemical; (ii) a second dose of 2-mercaptoethane sulfonic acid, or a salt or solvate thereof, is administered intravenously to the subject about 2 hours after the subject was exposed to the toxic inhaled chemical; and (iii) a third dose of 2-mercaptoethane sulfonic acid, or a salt or solvate thereof, is administered intravenously to the subject about 6 hours after the subject was exposed to the toxic inhaled chemical.

In certain embodiments, (i) a first dose of 2-mercaptoethane sulfonic acid, or a salt or solvate thereof, is administered intravenously to the subject within about 20 minutes after the subject was exposed to the toxic inhaled chemical; (ii) a second dose of 2-mercaptoethane sulfonic acid, or a salt or solvate thereof, is administered orally to the subject about 2 hours after the subject was exposed to the toxic inhaled chemical; and (iii) a third dose of 2-mercaptoethane sulfonic acid, or a salt or solvate thereof, is administered orally to the subject about 6 hours after the subject was exposed to the toxic inhaled chemical.

In certain embodiments, the method further comprises administering to the subject a therapeutically effective amount of a fibrinolytic agent.

In certain embodiments the fibrinolytic agent is tissue plasminogen activator (tPA) or an analog thereof.

In certain embodiments, between about 0.4 mg/kg to about 1.0 mg/kg tPA or its analog is administered to the subject.

In certain embodiments, tPA or its analog is administered to the subject about six hours to about 24 hours after the subject was exposed to the toxic inhaled chemical.

In certain embodiments, tPA or its analog is administered to the subject when the subject shows at least one of symptoms (i)-(v): (i) acutely worsening respiratory distress; (ii) acutely worsening hypoxemia; (iii) evidence of acutely worsening upper and/or lower airways obstruction selected from stridor, suprasternal retractions, wheezing, chest retractions, and combinations thereof; (iv) fibrinous casts, clots, or pseudomembranes; or (v) a blood-oxygen saturation≤85%.

In certain embodiments, tPA or its analog is administered to the subject via a bronchoscopy or an endotracheal tube.

In certain embodiments, the method improves survivability of the subject, peripheral arterial oxygen saturation in the subject, or a combination thereof.

In certain embodiments, the method prevents or decreases airway coagulation, cast formation, or a combination thereof in the subject.

In certain embodiments, the subject is a mammal. In certain embodiments, the subject is a human subject.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 shows the impact of sodium 2-mercaptoethane sulfonate (MESNA) treatment (300 mg/kg/dose given by intraperitoneal route at 0.5, 4, and 8 hours after 30 min exposure to methyl isocyanate (Bhopal agent, MIC; 500 ppm)) in rats monitored for 24 hours. There was a >30% increase in survival in rats given MESNA versus no treatment (p<0.0005).

FIG. 2 shows the results of an experiment wherein rats exposed to MIC (500 ppm×30 min by inhalation) were treated with tPA, MESNA, or a combination thereof to evaluate the effects of the different therapies. The experiment further included two types of controls, untreated rats and controls given two placebos (saline and PBS).

FIG. 3 depicts that MESNA treatment (3 doses) improves survival 80-100% at 24 hours after high-dose sulfur mustard (SM) inhalation when compared to placebo treatment (solvent) or no treatment, even when the MESNA treatment is delayed by 2 hours after exposure.

FIG. 4 shows that MESNA treatment given “early” (20 min) or “late” (2 h) after sulfur mustard vapor inhalation increases survival to 24 hours as compared to placebo treatment (solvent) or no treatment.

FIG. 5 demonstrates the recent re-establishment of the acute high-dose sulfur mustard inhalation toxicity (LD₁₀₀) model, in which a range of doses (3.8-4.2 mg/kg/exposure) were tested and consistent 100% mortality was confirmed, validating the model. This model re-validation was done prior to efficacy testing for MESNA.

FIG. 6 depicts that MESNA-treated SM inhalation (LD₁₀₀) rats survived >2-7 days after acute high-dose vs<24 h for two control groups. Furthermore, several MENSA treated animals survived >15 days and were then electively terminated. MESNA treatments were only given in the first 8 hours after exposure.

FIG. 7 shows that oxygen delivery to the tissues, assessed by pulse oximetry, was greater in rats exposed to sulfur mustard and treated with MESNA, as compared to those given no treatment (‘SM’) or placebo at 0-24 h, and at 0-5 days. (Rats surviving to 5 days also survived to 15 days at which time the study was electively terminated).

FIG. 8 shows the results of arterial blood gases, an important test of gas exchange and lung function. In rats electively euthanized when pulse oximetry was <70%, and in those surviving to 24 h, arterial pH, PaO₂, and PaCO₂, all were improved or tended to be improved in rats treated early or late with MESNA, relative to those values for rats given no treatment or placebo.

FIG. 9 shows airway cast scores (degree of airway occlusion by airway fibrin casts, plugs, or clots assessed at airway microdissection) in rats treated with MESNA early or late. A score of 7 indicates that the airways in that composite are 100% occluded.

FIG. 10 shows airway cast scores for rats succumbing after sulfur mustard exposure at a) up to 24 hours after exposure (Study #1, left panel), orb) up to 7 days (Study #2, right panel).

FIG. 11 shows the pathways of methionine and cysteine metabolism that may be relevant to actions of MESNA. Gas chromatography-mass spectrometry (GC/MS) has been used to measure all of the metabolites in these pathways. Formation of cysteine by transsulfuration pathway and its entry into cells rate-limits the subsequent formation of the critical antioxidant glutathione (GSH).

FIG. 12 is a table displaying the changes in the metabolites of FIG. 11 in rats injected with MESNA (150 mg/kg, one dose), as measured at 24 hours after injection.

FIG. 13 shows impact of sulfur mustard (SM) on transepithelial electrical resistance (TEER) and that MESNA reversed a significant part of the decline in TEER due to SM. SM and MESNA compounds were mixed immediately prior to the application of these compounds to the apical surface of cultured human nasal airway epithelia (HNE).

FIG. 14 shows dose-response to nitrogen mustard (NM) over time (120 h). 250 micromolar NM exposure for 24 hours results in a large decrease in TEER, while 62.5 micromolar had minimal effect. 1000 micromolar NM exposure destroyed the HNE, but 250 micromolar NM-exposed cells remained intact throughout the extended experimental timeline (120 h).

FIG. 15 shows that MESNA rescues airway epithelial cells from NM toxicity. Mixing 1000 micromolar MESNA with 250 micromolar NM immediately before addition to the apical surface inhibits the NM-induced reduction in TEER.

FIG. 16 shows effects of increasing CEES (‘half-mustard’) concentration exposure on TEER. Exposure to 6.4 mM CEES for 24 hours results in a large decrease in TEER, while 0.4 mM had minimal effect.

FIG. 17 shows effects of MESNA on half-mustard (CEES) disruption of TEER: Mixing equimolar (2.4 mM each) MESNA with CEES just before apical application to cells prevents TEER decline.

FIG. 18 is a graph showing results of reaction of MESNA (0-8 mM) or NM (0-96 mM) with Ellman reagent (DTNB) in a buffer. MESNA caused an absorbance increase of Ellman reagent (412 nm) over that range, and that absorbance was stable for >3 hr (left panel). By contrast, addition of NM without MESNA caused no increase in absorbance (right panel).

FIG. 19 is a graph showing that when the concentration of MESNA was held constant at 1.5 mM, increasing amount of NM was added, reactions were incubated for 20 mins or 3 h and then DTNB was added at the end of the reaction, there was a decline in absorbance (412 nm) over time. This suggests that a complex between MESNA and NM could be formed.

FIG. 20 is a schematic showing that the reaction between MESNA and HN2 (NM) likely proceeds from a 1:1 MESNA:NM reaction to a 2:1 MESNA:NM product (the larger, 363 Da molecule shown at far right).

FIG. 21A is the mass spectrum (product ion scan) of the 363 m/z fragment corresponding to a 2:1 MESNA:NM product from the reaction of MESNA and NM in aqueous solution. FIG. 21B illustrates assignment of the most likely fragments of the MESNA:NM product.

### DETAILED DESCRIPTION OF THE DISCLOSURE

Disclosed herein, according to various embodiments, is a system, method, process, and/or treatment for inhibition of mortality by exudative lung injury due to toxic gas inhalation. In some embodiments, the disclosure relates to using 2-mercaptoethane sulfonic acid, or a salt or solvate thereof, to inhibit death from toxic gas inhalation. In certain embodiments, 2-mercaptoethane sulfonic acid, or a salt or solvate thereof, is sodium 2-mercaptoethane sulfonate (MESNA). In various embodiments, MESNA may also help to decrease plasma cysteine, supporting the concept that MESNA drove cysteine into cells in order to protect them. Given the potential use of toxic inhaled chemicals as weapons of war or terrorism, this simple antidote could be stockpiled and used to protect large populations.

MESNA is an antioxidant drug, according to various embodiments. MESNA is a relatively inexpensive drug and is one of the 100 most used drugs in the world. As disclosed herein, MESNA may be administered to a patient to prevent mortality due to toxic gas inhalation, according to various embodiments. In various situations, the drug may decrease mortality by 30% after inhalation of methyl isocyanate, Bhopal agent. In various embodiments, after inhalation of sulfur mustard (mustard gas, 4.2 mg/kg), MESNA decreased mortality from 90-100% to 0-10% when compared to placebo-treated or untreated controls. In some embodiments, airway cast formation was not affected. Other sulfhydryl drugs may be ineffective.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

MESNA and Mesna are used interchangeably herein to refer to sodium 2-mercaptoethane sulfonate, or any other salt of 2-mercaptoethane sulfonate.

Mesnex is used herein to refer to an intravenous composition comprising sodium 2-mercaptoethane sulfonate. In some embodiments, the composition further comprises EDTA and/or benzyl alcohol. In some embodiments, the composition comprises an amount of sodium hydroxide to achieve a pH of between about 7.5-8.5.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

In one aspect, the present disclosure relates to a composition comprising 2-mercaptoethane sulfonic acid, or a salt or solvate thereof. In some embodiments, composition comprises sodium 2-mercaptoethane sulfonate. In certain embodiments, the composition comprises a therapeutically effective amount of 2-mercaptoethane sulfonic acid, or a salt or solvate thereof in order to treat TIC exposure in a subject. In certain embodiments, the TIC is sulfur mustard, chlorine, methyl isocyanate, methyl mercaptan, or a combination thereof. In certain embodiments, the composition comprises a therapeutically effective amount of 2-mercaptoethane sulfonic acid, or a salt or solvate thereof in order to treat exposure to sulfur mustard, methyl isocyanate, or a combination thereof, in the subject.

In some embodiments, the therapeutically effective amount of 2-mercaptoethane sulfonic acid, or a salt or solvate thereof, is contained inside of a nanoparticle. The nanoparticle may comprise any components known to a person of skill in the art to form nanoparticles that are also known or believed to be safe to administer to a human or animal subject. In certain embodiments, the nanoparticle does not comprise metallic components and is a non-metallic nanoparticle. In certain embodiments, the nanoparticle comprises a polymer. Exemplary polymers include, but are not limited to polyethylene glycol (PEG), polypropylene glycol (PPG), polylactic acid (PLA), polyglycolic acid (PGA), (poly(lactic-co-glycolic) acid (PLGA), and combinations thereof. In some embodiments, the nanoparticle surface is coated with a polymer. Exemplary polymers are described above. Therefore, in some embodiments, the nanoparticle comprises 2-mercaptoethane sulfonic acid, or a salt or solvate thereof, contained inside of a polymer shell. In another embodiment, the nanoparticle comprises a liposome. In certain embodiments, the nanoparticle is a liposome which contains 2-mercaptoethane sulfonic acid, or a salt or solvate thereof. In some embodiments, the liposome nanoparticle contains a composition comprising 2-mercaptoethane sulfonic acid, or a salt or solvate thereof.

Although not wishing to be limited by theory, it is believed that the nanoparticle provides sustained, continuous, delayed, or time-released delivery of 2-mercaptoethane sulfonic acid, or a salt or solvate thereof, to the subject. Although not wishing to be limited by theory, it is believed that the nanoparticle may prolong the circulating half-life, decrease allergic responses, and/or decrease irritant responses when 2-mercaptoethane sulfonic acid, or a salt or solvate thereof, is administered to a subject. In some embodiments, the long circulating half-life and controlled delivery of 2-mercaptoethane sulfonic acid, or a salt or solvate thereof, allows the subject to be treated for a TIC exposure with fewer administrations of nanoparticles comprising 2-mercaptoethane sulfonic acid, or a salt or solvate thereof, when compared with compositions which are not nanoparticles. Therefore, in certain embodiments, the administration of nanoparticles comprising 2-mercaptoethane sulfonic acid, or a salt or solvate thereof, could decrease the complexity of management of TIC disasters, for example in a mass-exposure incident. In some embodiments, the nanoparticle provides sustained, continuous, delayed, or time-released delivery of 2-mercaptoethane sulfonic acid, or a salt or solvate thereof, when a composition comprising the nanoparticle is administered to the subject subcutaneously or intramuscularly.

In certain embodiments, the composition comprises a pharmaceutically acceptable carrier. Exemplary pharmaceutically acceptable carriers are described elsewhere herein. In certain embodiments, the composition comprises one or more liquid pharmaceutically acceptable carriers such that the composition may be administered intravenously. In some embodiments, the intravenous composition comprises 2-mercaptoethane sulfonic acid, or a salt or solvate thereof, in a concentration of between about 10 mg/ml to about 500 mg/ml, about 10 mg/ml to about 450 mg/ml, about 10 mg/ml to about 400 mg/ml, about 10 mg/ml to about 350 mg/ml, about 10 mg/ml to about 300 mg/ml, about 10 mg/ml to about 250 mg/ml, about 10 mg/ml to about 200 mg/ml, about 10 mg/ml to about 150 mg/ml, about 50 mg/ml to about 150 mg/ml, or about 100 mg/ml. In some embodiments, the 2-mercaptoethane sulfonate salt is sodium 2-mercaptoethane sulfonate. In certain embodiments, the intravenous composition comprises edetate disodium (EDTA). In certain embodiments, the intravenous composition comprises EDTA in a concentration of about 0.01 mg/ml to about 5.0 mg/ml, about 0.01 mg/mg to about 4.5 mg/ml, about 0.01 mg/ml to about 4.0 mg/ml, about 0.01 mg/ml to about 3.5 mg/ml, about 0.01 mg/ml to about 3.0 mg/ml, about 0.01 mg/ml to about 2.5 mg/ml, about 0.01 mg/ml to about 2.0 mg/ml, about 0.01 mg/ml to about 1.5 mg/ml, about 0.01 mg/ml to about 1.0 mg/ml, about 0.1 mg/ml to about 1.0 mg/ml, about 0.1 mg/ml to about 0.5 mg/ml, or about 0.25 mg/ml. In certain embodiments, the intravenous composition comprises a base to maintain a physiological pH. In some embodiments, the base is sodium hydroxide. In certain embodiments, the intravenous composition has a pH of between about 7.5 to about 8.5. In certain embodiments, the intravenous composition comprises a preservative. In some embodiments, the preservative is benzyl alcohol. In certain embodiments, the intravenous composition comprises benzyl alcohol in a concentration of about 1.0 mg/ml to about 50 mg/ml, about 1.0 mg/ml to about 45 mg/ml, about 1.0 mg/ml to about 40 mg/ml, about 1.0 mg/ml to about 35 mg/ml, about 1.0 mg/ml to about 30 mg/ml, about 1.0 mg/ml to about 25 mg/ml, about 1.0 mg/ml to about 20 mg/ml, about 1.0 mg/ml to about 15 mg/ml, about 5.0 mg/ml to about 15 mg/ml, or about 10 mg/ml. In some embodiments, the intravenous composition comprises about 100 mg/ml sodium 2-mercaptoethane sulfonate, about 0.25 mg/ml EDTA, about 10.4 mg/ml benzyl alcohol, and an amount of sodium hydroxide to achieve a pH of between about 7.5-8.5.

In other embodiments, the composition comprises one or more solid pharmaceutically acceptable carriers such that the composition may be administered orally. In some embodiments, the oral composition comprises 2-mercaptoethane sulfonic acid, or a salt or solvate thereof, in a concentration of between about 50 mg to about 800 mg, about 50 mg to about 750 mg, about 50 mg to about 700 mg, about 50 mg to about 650 mg, about 100 mg to about 650 mg, about 200 mg to about 650 mg, about 250 mg to about 650 mg, about 250 mg to about 600 mg, about 250 mg to about 550 mg, about 250 mg to about 500 mg, about 300 mg to about 500 mg, about 350 mg to about 500 mg, about 350 mg to about 450 mg, or about 400 mg. In certain embodiments, the 2-mercaptoethane sulfonate salt is sodium 2-mercaptoethane sulfonate. In certain embodiments, the oral composition comprises one or more excipients. In certain embodiments, the oral composition comprises one or more of the following excipients: calcium phosphate, cornstarch, hydroxypropylmethylcellulose, lactose, magnesium stearate, microcrystalline cellulose, polyethylene cellulose, povidone, simethicone, and titanium dioxide. In certain embodiments, the oral composition comprises each of calcium phosphate, cornstarch, hydroxypropylmethylcellulose, lactose, magnesium stearate, microcrystalline cellulose, polyethylene cellulose, povidone, simethicone, and titanium dioxide. In some embodiments, the oral composition comprises about 400 mg sodium 2-mercaptoethane sulfonate and each of calcium phosphate, cornstarch, hydroxypropylmethylcellulose, lactose, magnesium stearate, microcrystalline cellulose, polyethylene cellulose, povidone, simethicone, and titanium dioxide. In some embodiments, the oral composition is a pill, a tablet, a capsule, or a gelcap.

In some embodiments, the composition comprises 2-mercaptoethane sulfonic acid, or a salt or solvate thereof, and an additional pharmaceutically active compound. The additional pharmaceutically active compound can be any compound known to a person of skill in the art to aid in the treatment of a subject who has been exposed to a TIC. In certain embodiments, the additional pharmaceutically active compound is a fibrinolytic agent. Although not wishing to be limited by theory, it is believed that airway occlusion with fibrin plugs can still occur during treatment of a subject with 2-mercaptoethane sulfonic acid, or a salt or solvate thereof. Therefore, a fibrinolytic agent may be added to the composition. In certain embodiments, the fibrinolytic agent is tissue plasminogen activator (tPA), urokinase-type plasminogen activator (uPA), or a combination thereof. In certain embodiments, the additional pharmaceutically active compound is thioredoxin or a protein comprising a thioredoxin active-site, including but not limited to, mutant or foreshortened protein/peptide thioredoxin analogs containing the active site. In some embodiments, the protein comprises a thioredoxin active site in a reduced state. In certain embodiments, the additional pharmaceutically active compound is an inhibitor of, or an antibody against, plasminogen activator inhibitor-1 (PAI-1). Although not wishing to be limited by theory, it is believed that PAI-1 may be induced after exposure to sulfur mustard or MIC. In some embodiments, PAI-1 is induced after inhalation of sulfur mustard and/or MIC.

In certain embodiments, the composition comprises a chelator. In some embodiments, the composition is an intravenous composition comprising a chelator. In some embodiments, the chelator is EDTA. Therefore, in some embodiments, the composition is an intravenous composition comprising a concentration of EDTA described elsewhere herein. In certain embodiments, EDTA functions to chelate iron. In another embodiment, the chelator is another iron chelator such as deferoxamine (desferrioxamine, DFOA). In certain embodiments, the composition is an intravenous composition comprising a concentration of DFOA which is identical to the concentrations of EDTA described elsewhere herein. In other embodiments, the composition is an oral composition described elsewhere herein wherein the oral composition further comprises a chelator such as EDTA or DFOA. Although not wishing to be limited by theory, it is believed that, in the absence of a chelator, 2-mercaptoethane sulfonic acid, or a salt or solvate thereof, will auto-oxidase. Therefore, in some embodiments, the compositions described elsewhere herein comprise an amount of chelator that is sufficient to prevent substantial auto-oxidation of the 2-mercaptoethane sulfonic acid, or salt or solvate thereof. In some embodiments, DFOA has other off-target effects, including, but not limited to, such as activation of hypoxia-inducible factors (HIFs), inhibition of essential mitochondrial enzymes (aconitase), inhibition of mitochondrial respiration, and combinations thereof. Therefore, in some embodiments when the composition comprises a DFOA chelator, the off-target effects of DFOA should be managed. In some embodiments, the off-target effects can be managed by selecting a concentration of DFOA which minimizes one or all of the off-target effects while still preventing substantial (or a portion of) the auto-oxidation of 2-mercaptoethane sulfonic acid, or a salt or solvate thereof.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Methods

In another aspect, the present disclosure relates to a method of treating a subject exposed to a toxic inhaled chemical, the method comprising administering to the subject a therapeutically effective amount of 2-mercaptoethane sulfonic acid, or a salt or solvate thereof.

The therapeutically effective amount of 2-mercaptoethane sulfonic acid, or a salt or solvate thereof, may be administered to the subject as a component of a composition. In some embodiments, the composition comprises one or more pharmaceutically acceptable carriers described elsewhere herein. In some embodiments, the composition comprises one or more additional pharmaceutically active compounds described elsewhere herein. In some embodiments, the composition comprises nanoparticles containing 2-mercaptoethane sulfonic acid, or a salt or solvate thereof. In certain embodiments, the 2-mercaptoethane sulfonic acid, or salt or solvate thereof, is sodium 2-mercaptoethane sulfonate.

The therapeutically effective amount of 2-mercaptoethane sulfonic acid, or a salt or solvate thereof, may be administered to the subject using any route of administration known to a person of skill in the art. In some embodiments, 2-mercaptoethane sulfonic acid, or salt or solvate thereof, is administered to the subject via one of the following routes: oral, intravenous, subcutaneous, intra-osseus, intramuscular, intraperitoneal, intratracheal, cutaneous, intra-ocular, or a combination thereof. In certain embodiments, 2-mercaptoethane sulfonic acid, or salt or solvate thereof, is administered to the subject orally, intravenously, subcutaneously, intramuscularly, or a combination thereof. In some embodiments, 2-mercaptoethane sulfonic acid, or salt or solvate thereof, is administered to the subject orally as a pill, tablet, capsule, or gelcap. Although not wishing to be limited by theory, it is believed that sulfur mustard affects the lungs (large and small airways), corneas, and skin most severely. That means that any and all of the above preparations may be useful in treating subjects exposed to this TIC, based on the individual condition of each subject.

2-mercaptoethane sulfonic acid, or a salt or solvate thereof, can be administered to the subject in any amount that provides a therapeutic effect in the subject after the subject has been exposed to a TIC. In certain embodiments, the therapeutic effect is an improvement in the survivability of the subject, an improvement in peripheral arterial oxygen saturation in the subject, or a combination thereof. In some embodiments, the TIC is sulfur mustard, methyl isocyanate, or a combination thereof. Therefore, in some embodiments, an amount of 2-mercaptoethane sulfonic acid, or a salt or solvate thereof, is administered to a subject to improve the subject's survivability, peripheral arterial oxygen saturation, or a combination thereof, after the subject has been exposed to sulfur mustard, methyl isocyanate, or a combination thereof. In some embodiments, between about 120 mg/m² to about 720 mg/m² of 2-mercaptoethane sulfonic acid, or a salt or solvate thereof, is administered to the subject. In certain embodiments wherein 2-mercaptoethane sulfonic acid, or a salt or solvate thereof, is administered intravenously to the subject, about 120 mg/m² to about 360 mg/m² of 2-mercaptoethane sulfonic acid, or a salt or solvate thereof, is administered to the subject. In other embodiments wherein 2-mercaptoethane sulfonic acid, or a salt or solvate thereof, is administered orally to the subject, about 240 mg/m² to about 720 mg/m² of 2-mercaptoethane sulfonic acid, or a salt or solvate thereof, is administered to the subject. In certain embodiments, about 120 mg/m² to about 360 mg/m² sodium 2-mercaptoethane is administered intravenously to a subject who has been exposed to sulfur mustard, methyl isocyanate, or a combination thereof. In other embodiments, about 240 mg/m² to about 720 mg/m² sodium 2-mercaptoethane is administered orally to a subject who has been exposed to sulfur mustard, methyl isocyanate, or a combination thereof. In some embodiments, between about 5 mg/kg to about 100 mg/kg, about 5 mg/kg to about 90 mg/kg, about 10 mg/kg to about 85 mg/kg, about 15 mg/kg to about 80 mg/kg, about 20 mg/kg to about 80 mg/kg, or about 25 mg/kg to about 75 mg/kg of 2-mercaptoethane, or a salt or solvate thereof, is administered to the subject. In certain embodiments, between about 25 mg/kg to about 75 mg/kg of 2-mercaptoethane, or a salt or solvate thereof, is administered to the subject orally, intravenously, or using a combination thereof. In certain embodiments, between about 25 mg/kg to about 75 mg/kg of 2-mercaptoethane, or a salt or solvate thereof, is administered to a subject who has been exposed to sulfur mustard, methyl isocyanate, or a combination thereof, wherein the administration is oral, intravenous, or using a combination thereof.

Administration of 2-mercaptoethane sulfonic acid, or a salt or solvate thereof, can occur at any time after the subject has been exposed to the TIC. In certain embodiments, 2-mercaptoethane sulfonic acid, or a salt or solvate thereof, is administered to the subject immediately to about ten hours after the subject was exposed to the toxic inhaled chemical. In some embodiments, 2-mercaptoethane sulfonic acid, or a salt or solvate thereof, is administered to the subject within about 30 minutes after the subject was exposed to the toxic inhaled chemical. In other embodiments, 2-mercaptoethane sulfonic acid, or a salt or solvate thereof, is administered to the subject about two hours to about eight hours after the subject was exposed to the toxic inhaled chemical. Although not wishing to be limited by theory, it is believed that administration of 2-mercaptoethane sulfonic acid, or a salt or solvate thereof, should preferably occur as soon as possible after the subject's exposure to the TIC and within about 30 minutes of the subject's exposure to the TIC. However, if necessary, the administration of 2-mercaptoethane sulfonic acid, or a salt or solvate thereof, can be delayed to about 8 hours after the subject's exposure to the TIC. Delayed administration of 2-mercaptoethane sulfonic acid, or a salt or solvate thereof, will still provide an improvement survivability of the subject, an improvement in peripheral arterial oxygen saturation in the subject, or a combination thereof, when compared to subjects who are not treated for exposure to the TIC.

In certain embodiments, 2-mercaptoethane sulfonic acid, or a salt or solvate thereof, is administered to the subject in multiple doses. In certain embodiments, the doses described elsewhere herein are administered to the subject in a first, second, and third dose. In certain embodiments the first dose of 2-mercaptoethane sulfonic acid, or a salt or solvate thereof, is administered orally and/or intravenously to the subject within about 20 minutes after the subject was exposed to the toxic inhaled chemical. In certain embodiments, the second dose of 2-mercaptoethane sulfonic acid, or a salt or solvate thereof, is administered orally and/or intravenously to the subject about 4 hours after the subject was exposed to the toxic inhaled chemical. In certain embodiments, the third dose of 2-mercaptoethane sulfonic acid, or a salt or solvate thereof, is administered orally and/or intravenously to the subject about 8 hours after the subject was exposed to the toxic inhaled chemical.

In certain embodiments the first dose of 2-mercaptoethane sulfonic acid, or a salt or solvate thereof, is administered orally and/or intravenously to the subject within about 2 hours after the subject was exposed to the toxic inhaled chemical. In certain embodiments, the second dose of 2-mercaptoethane sulfonic acid, or a salt or solvate thereof, is administered orally and/or intravenously to the subject about 4 hours after the subject was exposed to the toxic inhaled chemical. In certain embodiments, the third dose of 2-mercaptoethane sulfonic acid, or a salt or solvate thereof, is administered orally and/or intravenously to the subject about 8 hours after the subject was exposed to the toxic inhaled chemical.

In certain embodiments, wherein 2-mercaptoethane sulfonic acid, or a salt or solvate thereof, is administered intravenously to the subject, a first dose is administered intravenously to the subject immediately to within about 20 minutes after the subject was exposed to the toxic inhaled chemical, a second dose is administered intravenously to the subject about 4 hours after the subject was exposed to the toxic inhaled chemical, and a third dose is administered intravenously to the subject about 8 hours after the subject was exposed to the toxic inhaled chemical. In other embodiments, wherein 2-mercaptoethane sulfonic acid, or a salt or solvate thereof, is administered orally to the subject, a first dose is administered orally to the subject immediately to within about 20 minutes after the subject was exposed to the toxic inhaled chemical, a second dose is administered orally to the subject about 2 hours after the subject was exposed to the toxic inhaled chemical, and a third dose is administered orally to the subject about 6 hours after the subject was exposed to the toxic inhaled chemical. In yet other embodiments, a first dose of 2-mercaptoethane sulfonic acid, or a salt or solvate thereof, is administered intravenously to the subject immediately to within about 20 minutes after the subject was exposed to the toxic inhaled chemical, a second dose is administered orally to the subject about 2 hours after the subject was exposed to the toxic inhaled chemical, and a third dose is administered orally to the subject about 6 hours after the subject was exposed to the toxic inhaled chemical.

In some embodiments, the method further comprises the step of administering to the subject a therapeutically effective amount of an additional pharmaceutically active compound. Exemplary additional pharmaceutically active compounds described elsewhere herein. In certain embodiments, the additional pharmaceutically active compound is thioredoxin or a protein comprising a thioredoxin active-site. In other embodiments, the additional pharmaceutically active compound is a fibrinolytic agent, such as, for example, tissue plasminogen activator (tPA) or an analog thereof. In certain embodiments, between about 0.01 mg/kg to about 5.0 mg/kg, about 0.01 mg/kg to about 4.0 mg/kg, about 0.01 mg/kg to about 3.0 mg/kg, about 0.01 mg/kg to about 2.0 mg/kg, about 0.1 mg/kg to about 1.5 mg/kg, or about 0.4 mg/kg to about 1.0 mg/kg tPA or its analog is administered to the subject. In some embodiments, tPA or its analog is administered to the subject following administration of 2-mercaptoethane sulfonic acid, or a salt or solvate thereof. In some embodiments, tPA or its analog is administered to the subject following administration the third dose of 2-mercaptoethane sulfonic acid, or a salt or solvate thereof. In another embodiment, tPA or its analog is administered to the subject between the first dose and second dose of 2-mercaptoethane sulfonic acid, or a salt or solvate thereof. In yet another embodiment, tPA or its analog is administered to the subject between the second dose and third dose of 2-mercaptoethane sulfonic acid, or a salt or solvate thereof. In yet another embodiment, tPA or its analog is administered to the subject before the first dose of 2-mercaptoethane sulfonic acid, or a salt or solvate thereof. In certain embodiments, tPA or its analog is administered to the subject between about 6 hours and about 24 hours after the subject has been exposed to the toxic inhaled chemical. In certain embodiments, tPA or its analog is administered to the subject when the subject shows at least one of symptoms (i)-(v): (i) acutely worsening respiratory distress, (ii) acutely worsening hypoxemia, (iii) evidence of acutely worsening upper and/or lower airways obstruction selected from stridor, suprasternal retractions, wheezing, chest retractions, and combinations thereof, (iv) fibrinous casts, clots, or pseudomembranes, or (v) a blood-oxygen saturation≤85%. In some embodiments, tPA or its analog is administered to the subject when the subject shows at least one of symptoms (i)-(v) following administration of 2-mercaptoethane sulfonic acid, or a salt or solvate thereof. In certain embodiments, tPA or its analog is administered to the subject via a bronchoscopy or an endotracheal tube. In some embodiments wherein the blood-oxygen saturation in the subject is 85% or less following administration of tPA or its analog, a second dose of tPA is administered to the subject. In other embodiments, wherein administration of tPA or its analog improves blood-oxygen saturation in the subject such that it is greater than 85%, a second dose of tPA is administered to the subject when the subject's blood-oxygen saturation falls to 85% or less. In certain embodiments, administration of tPA or its analog improves one or more of symptoms (i)-(v) in the subject. In some embodiments, administration of tPA or its analog prevents or decreases airway coagulation, cast formation, or a combination thereof in the subject.

In certain embodiments, the subject is a mammal. In certain embodiments, the subject is a human.

### Pharmaceutical Compositions and Formulations

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or any combinations of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, microvesicles, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the disclosure are administered to the patient in dosages that range from one to six times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

Compounds of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 μg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, PA (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intraosseous (including, but not limited to, intra-tibial and intrasternal) injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, other anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically- or naturally derived.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the compositions and/or formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Mesnex as a Critical Care Rescue Medical Countermeasure for Toxic Gas Inhalation

MESNA (Mesnex (Baxter); 2-mercaptoethane sulfonate Na) is an organosulfur-containing small molecule whose chemoprotective actions occur by altering breakdown products of cyclophosphamide and ifosfamide, particularly acrolein, in the urine, thereby making them less toxic, and protecting the bladder from hemorrhagic injury. In addition to acrolein, MESNA's sulfhydryl group can react with other α,β-unsaturated carbonyl compounds, forming conjugates by Michael addition, then these are excreted. Mesnex was FDA-approved as an intravenous or orally-administered chemoprotective agent in 1988. MESNA is on the World Health Organization's List of Essential Medicines.

MESNA is water soluble. Metabolically, it is oxidized in the circulation. It was previously found that the associated reduction of cystine, GSSG and protein mixed disulfides in plasma was quite rapid (5 min), with a shift of the cysteine formed into cells and into the urine. Indeed, GC-MS showed that plasma cysteine decreased from 225 μM to 40 μM within 1 hour after dosing (150 mg/kg IP in rat). Using LC-MS/MS for analysis of rat plasma, MESNA was found to have a circulating half-life of 76.2 minutes when rats were administered MESNA at 300 mg/kg by intraperitoneal (IP) injection. MESNA was also found to have an elimination rate constant Kₑ of 0.0091, showing that the elimination of MESNA is very fast.

Medical countermeasures are needed for national priority chemical threats including toxic inhaled chemicals (TICs) such as sulfur mustard (SM, ‘mustard gas’), chlorine, methyl isocyanate (MIC; Bhopal agent), and methyl mercaptan. Of those listed above, 2 of the 4 remain among the top 5 on that list—SM and chlorine—based on threat assessment. SM is a blistering agent (‘vesicant’) that damages the skin, eye, and respiratory system. SM causes Tissue Factor-dependent activation of the clotting cascade both in the airways and in the circulation. These clots or casts obstruct the airways, just as those seen in children with ‘plastic bronchitis’ related to congenital heart disease. Acute SM-exposed rats were previously treated with tissue plasminogen activator (tPA, Alteplase, Genentech/Roche) given into the airway. This treatment relieved the obstruction and converted a 100% lethal to a 100% survival model for up to 48 hours. tPA has also been used in the porcine model with similar effect. Despite the lifesaving potential of tPA, it is a very expensive drug that can require general anesthesia and intratracheal instillation 2 to 6 times daily; and it is in that manner that it was used effectively in rats after high-level SM exposure (4 mg/kg, acute). It was also found that tPA had some efficacy after Bhopal agent (methyl isocyanate, MIC) inhalation, although never affording more than 50-60% survival. Ultimately, although tPA is efficacious, even lifesaving, after TIC exposure, its use in this scenario would require that the patient be sedated in the ICU and undergo tracheal intubation and bronchoscopy. Indeed it is fortunate that airway tPA can be delayed several hours after exposure and still be effective.

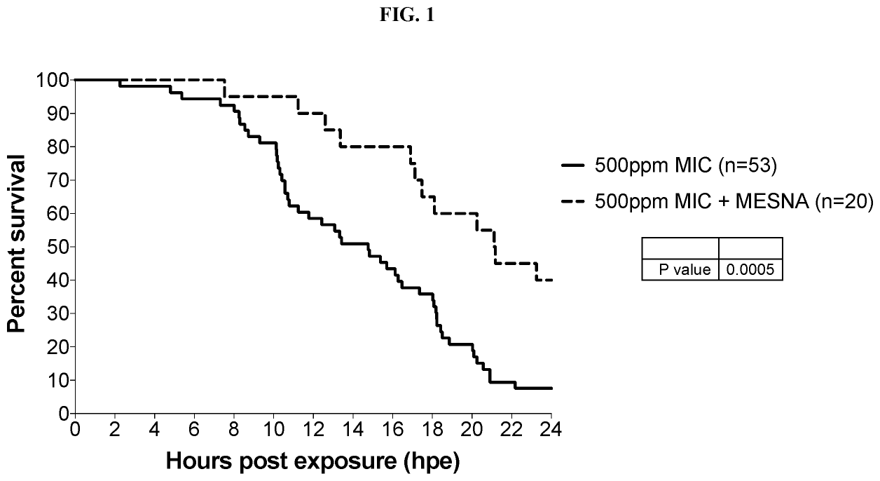

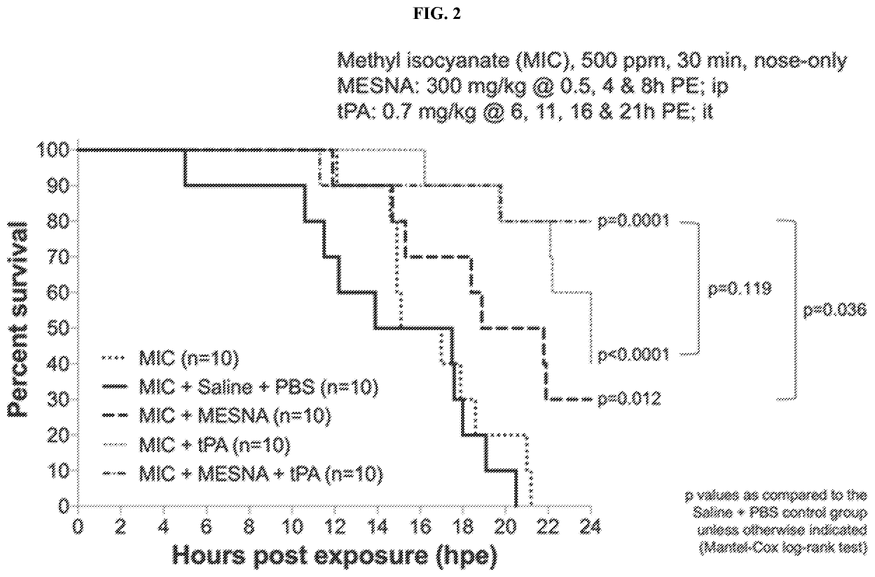

While investigating several potential adjunctive treatments, including antioxidants, it was found that MESNA (dosed at 0.5, 4, and 8 h after exposure), but not equimolar N-acetylcysteine nor glutathione, afforded a measurable but consistent 30% survival increase for MESNA treatment following MIC exposure versus 0% survival for controls (24 h) (FIG. 1), and combining MESNA+tPA afforded 80% survival after MIC exposure, an additive/synergistic benefit (FIG. 2). Specifically, FIG. 2 compares treatment with intratracheal tPA (tissue plasminogen activator) to treatment with MESNA, and treatment with both tPA and MESNA in rats exposed to MIC. tPA was dosed to rats (0.7 mg/kg/dose) starting at 6 hours after MIC exposure and re-dosed every 5 hours through the end of the 24-hour observation period. MESNA was dosed 3 times: at 0.5, 4, and 8 hours, and given ip. This MESNA regimen was found to be optimal/most beneficial based on previous efficacy studies of 3 sulfhydryl agents, with MESNA compared to N-acetylcysteine and reduced glutathione. It was found that animals in the ‘no treatment’ (‘MIC’) and in the double placebo group (‘MIC+Saline+PBS’) had 100% mortality at 20-22 hours. By contrast, survival was 30% at 24 hours with MESNA treatment alone (‘MIC+MESNA’; p=0.012). Likewise, with tPA treatment alone, survival was 40% (‘MIC+tPA’); p<0.0001) at 24 h. With the two treatments combined (‘MIC+MESNA+tPA’), survival was 80% at 24 h as compared to 0% in the double placebo group (p=0.0001). Notably, tPA+MESNA treatments combined was significantly superior to MESNA alone (p=0.036). Although not wishing to be limited by theory, these findings support the hypothesis that MESNA and tPA may act by different mechanisms.

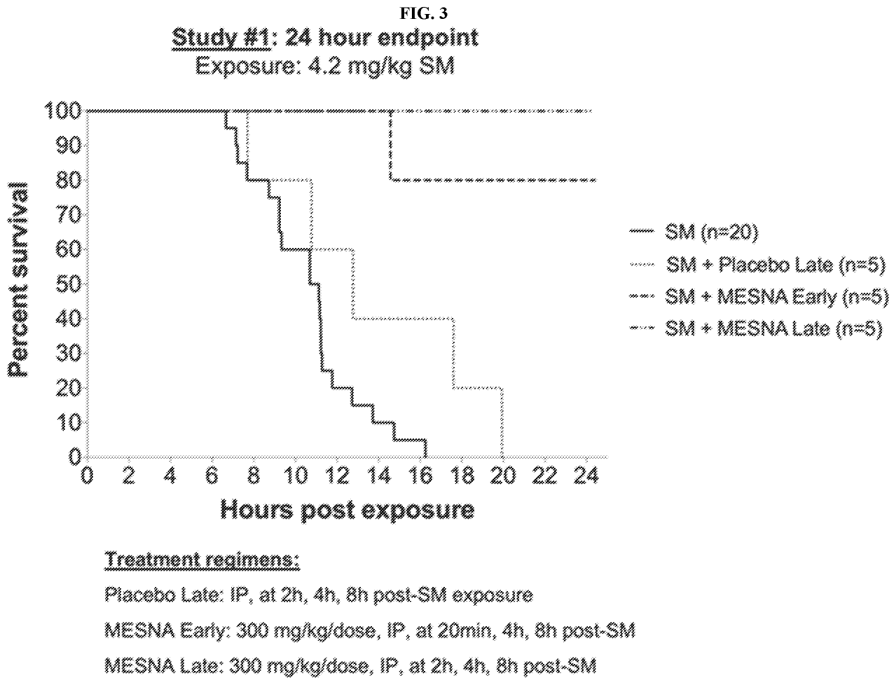

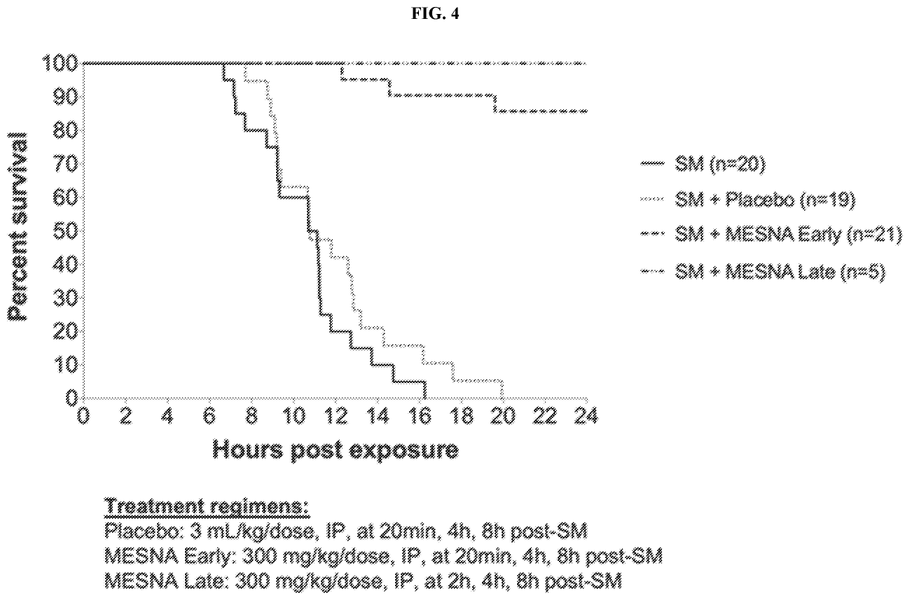

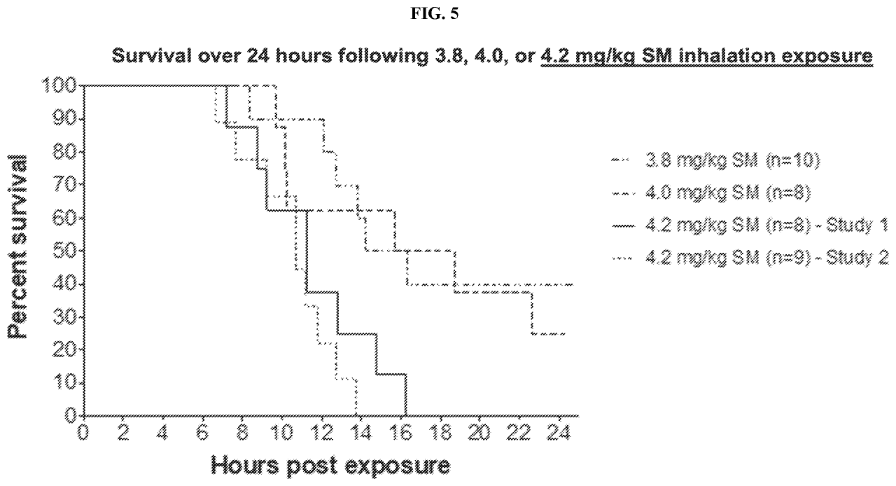

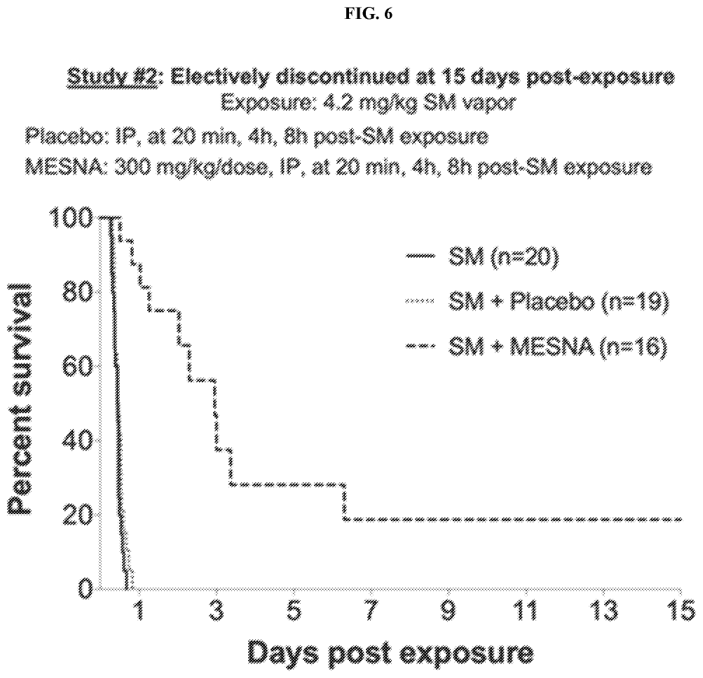

Like cyclophosphamide, SM is an alkylating agent. Therefore, this 3-dose MESNA regimen that had been developed was also tested to protect rats from cyclophosphamide toxicity in the high-dose SM inhalation model. Remarkably, rats treated with Mesnex only during the first 8 hours after SM (4.2 mg/kg, intraperitoneal) inhalation, have consistently shown 80-100% survival at 24 or 48 hours after exposure (FIG. 3 and FIG. 4). This compares to 100% mortality at 14-20 hours in untreated and placebo control groups (FIG. 3, FIG. 4, and FIG. 5). Further, there were multiple survivors at 48-360 h in the Mesnex-treated group (study discontinued at 360 h), even though these rats had received no Mesnex after 8 hours (FIG. 6). By contrast, both control groups had succumbed by <1 day (FIG. 6). In addition to these findings, it was found that, even when the initial dose of Mesnex was delayed 2 hours after exposure, survival was still 100% at 24 hours after exposure (versus 8-12 h survival in controls) (FIG. 3). Clearly, a delay of 2 hours in initiating MESNA treatment was not deleterious, as there was 100% survival in that group (FIG. 3). Future studies will examine the breadth of this therapeutic window, beyond 0.5-2 hours, in greater detail. Furthermore, FIG. 4 depicts that MESNA, given early (20 min) was superior to ‘placebo (early)’ (80% survival for MESNA-treated versus 0% survival to 24 hours in early placebo group) and a potential difference in survival when MESNA was given at 0.33 versus 2 h was seen, which will be the subject of a future study. Further studies will also assess: a) potential benefits of continuing MESNA therapy beyond the first 12 hours after exposure, and b) possible benefits of continuing tPA treatment, in combination with MESNA, beyond 24-48 hours.

Thus, Mesnex's lifesaving benefit after sulfur mustard inhalation is durable and sustained, does not require airway access, initial dosing can be delayed, and repeated dosing outside the acute phase may not be necessary. Therefore, in a mass casualty situation, it could be more rapidly administered to larger numbers of victims, and, as such, could be a more ideal antidote than tPA. Typically, FDA seeks MCMs that are: a) safe for all populations, including children; b) have a realistic treatment window, c) are easy to administer in a mass casualty situation, d) also may treat or prevent delayed or chronic effects of exposure, e) are inexpensive, and f) have a long shelflife Already Mesnex fulfills most of these.

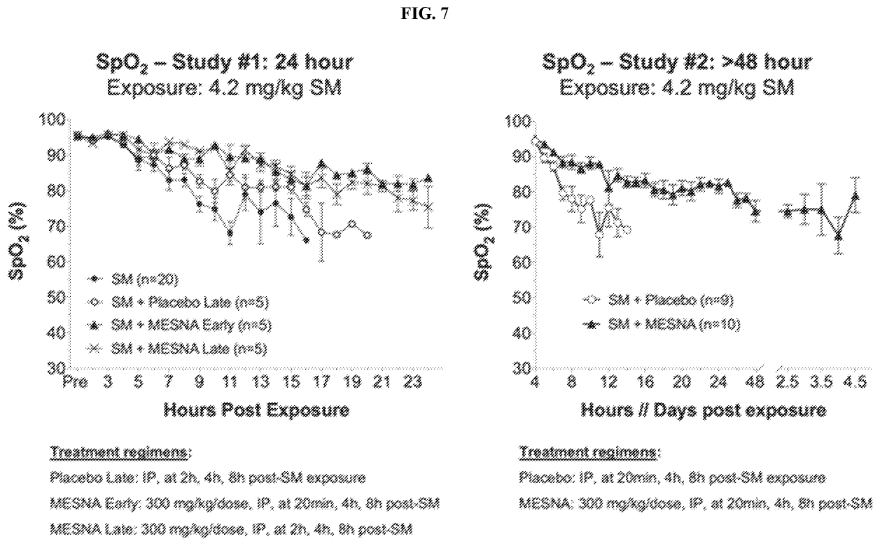

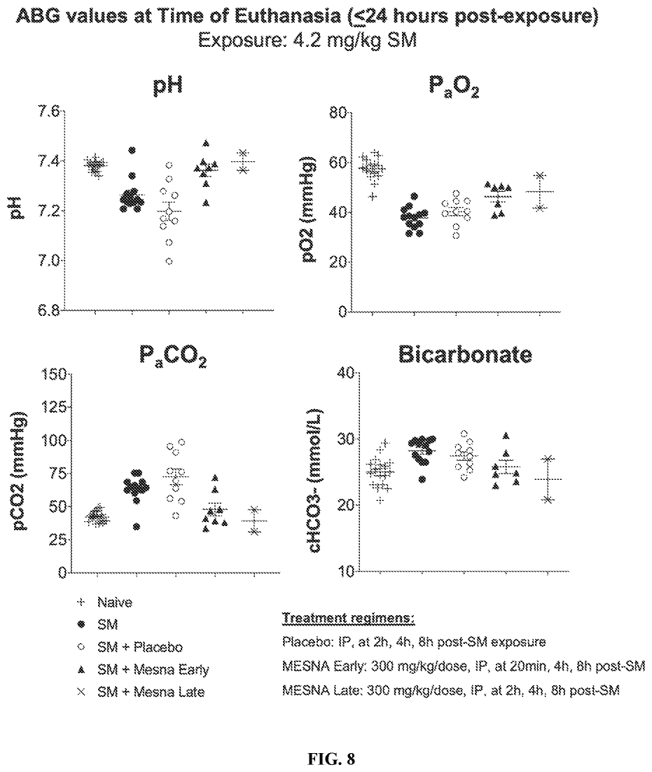

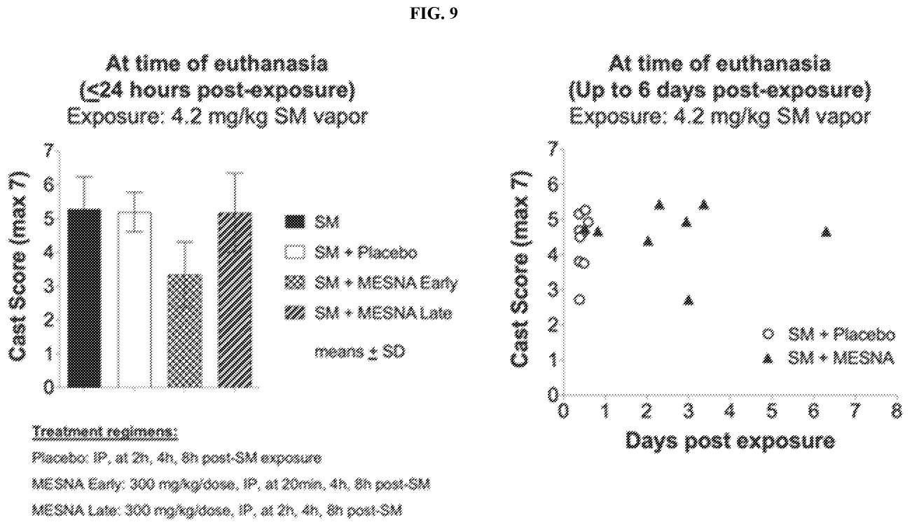

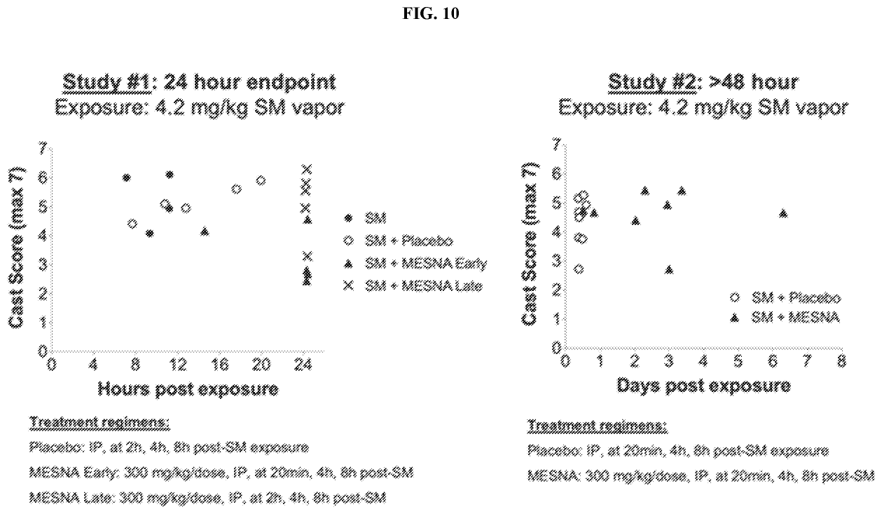

Studies demonstrated that Mesnex improves peripheral arterial oxygen saturation in rats (FIG. 7) as well as arterial blood gas (ABG) abnormalities (FIG. 8) compared to placebo and controls. Interestingly, airway obstructions by casts (clots), measured morphometrically, were not decreased in Mesnex-treated rats compared to no treatment nor placebo-treated controls (FIG. 9 and FIG. 10), suggesting that long term survival could be most consistent with combined treatment with tPA. Nonetheless, the durable protection afforded by early Mesnex treatment lasts for several days, and, in that regard, is unlike any rescue treatment we have developed in the laboratory or clinic. The data in FIG. 9 suggest that MESNA did not have lysis of fibrin casts as its primary mode of action, as the cast scores were not different in MESNA-treated versus control subjects. A caveat here, however, is that MESNA-treated rats were, as a rule, succumbing later than controls. Therefore, future studies will entail timed comparisons of the two groups to accurately obtain airway cast scores matched for time. The data in FIG. 10 again shows that the animals in the control and MESNA groups are succumbing, largely, at different times, and therefore future studies will entail timed comparisons of these two groups, likely with some early time points. Preliminary studies at 8 h after SM exposure show that cast scores were significantly lower in rats given MESNA versus those given diluent (placebo) or no treatment (not shown).

The mechanisms by which Mesnex acts are unclear but, without wishing to be limited by any theory, suspected to be at the initial stages of oxidative stress and/or DNA damage. Potential cytoprotective mechanisms of MESNA warrant further study.

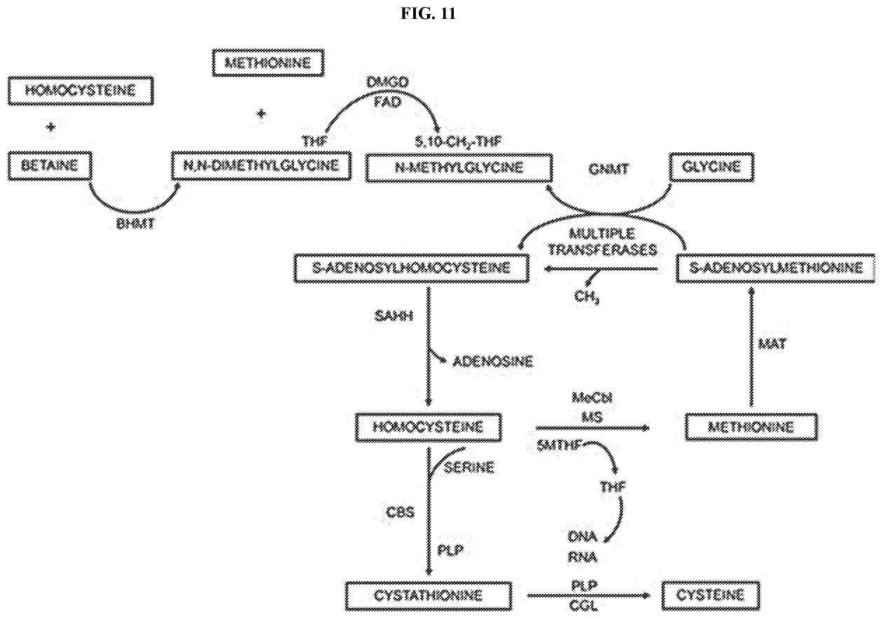

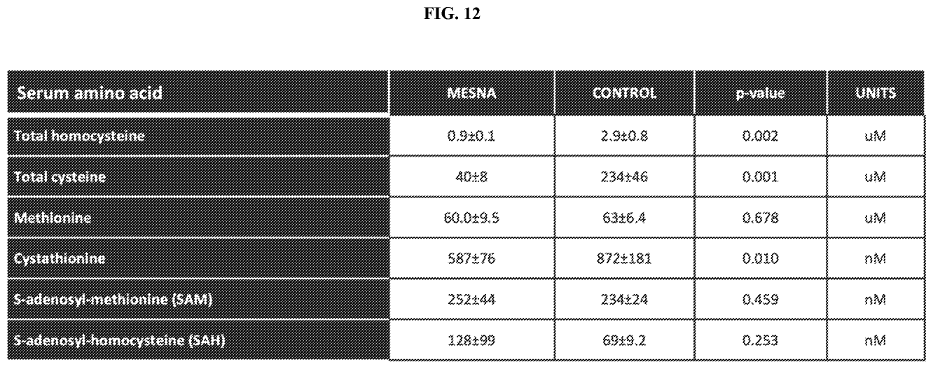

FIG. 11 depicts the pathways of methionine metabolism. The methylation of homocysteine to methionine and subsequent activation to S-adenosylmethionine is referred to as transmethylation, whereas the condensation of homocysteine and serine to form cystathionine and later cleavage to cysteine is referred to as transsulfuration. Cysteine is a rate-limiting substrate in the synthesis of glutathione. 5-CH3-THF, 5-methyltetrahydrofolate; CH3-Cbl, vitamin 12; THF, tetrahydrofolate. FIG. 12 depicts the effect of single MESNA injection (150 mg/kg IP) on serum sulfur amino acids (SAA; mean±SD) at 60 minutes after injection. SAA were measured by GC-MS. SAA of the transsulfuration pathway (total cysteine, total homocysteine, and cystathionine) were significantly different after MESNA injection, while those of the transmethylation pathway (methionine, SAM, and SAH) were not (p>0.05). Notably, liver SAA of the transsulfuration pathway, as well as methionine, were not different after MESNA (SAM and SAH were not measured). By far, the greatest change was in total cysteine in circulating plasma. This change in cysteine can be used as a biomarker of MESNA action. Although not wishing to be limited by theory, it is believed that this marked decline in plasma cysteine indicates that indeed cysteine was mobilized and driven into cells, and into the urine, by MESNA's action to reduce all accessible cysteines and form the oxidized species diMESNA.

When MESNA enters the bloodstream (rapidly with IV injection; more slowly with IP injection), it will promptly convert cystines (oxidized) to cysteines (reduced), oxidized glutathione (GSSG) to reduced glutathione (GSH), and protein-mixed disulfides to cysteines and reduced proteins. The latter will include critical endothelial cell surface proteins. Rebound decrease in cysteine in circulation will occur as cells rapidly take up MESNA. GSH will rapidly form, and thereby diminish injury by directly or indirectly binding to SM or MIC. Finally, overall lung exposure to reductants will induce signal transduction events to increase production of antioxidants such as manganese superoxide dismutase (MnSOD; SOD2), leading to additional protection.

### Example 2: Impact of Sulfur Mustard (SM) on Transepithelial Electrical Resistance (TEER) and Effect of MESNA

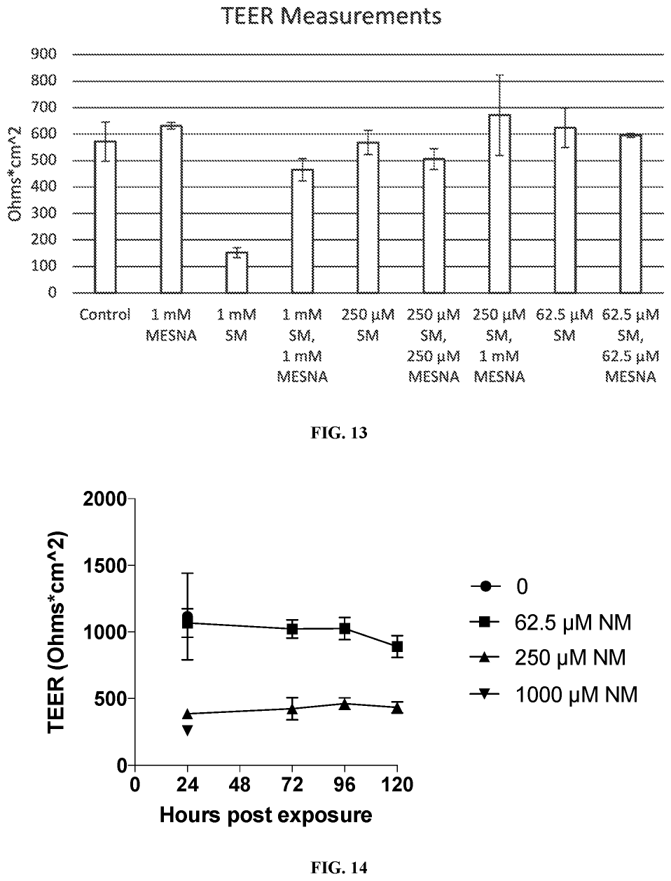

At first TEER (transepithelial electrical resistance) was examined across normal primary human nasal airway epithelial cells in wells containing 500 microliters of basal media and minimal apical fluid (<10 microliters at air-liquid interface). In control cells, TEER was 575±75 ohms*cm² (mean±SD) over a 24-hour observation period (FIG. 13). Adding 1 mM MESNA had little effect on TEER (625 ohms*cm²) over that duration. Apical addition of 62.5 uM SM, again, had little impact (620±80) as did 250 uM (575±50). By contrast, 1 mM SM decreased TEER to 150±25 ohms*cm² (third column from left). Addition of 1 mM SM+1 mM MESNA eliminated ⅔ of the decline in TEER caused by 1 mM SM, bringing TEER to 467±40 ohms*cm² (fourth column from left). Thus, TEER was a useful indicator of SM toxicity; and MESNA reversed a significant part of the decline in TEER due to SM.

### Example 3: Effects of NM on TEER and Reversal by MESNA

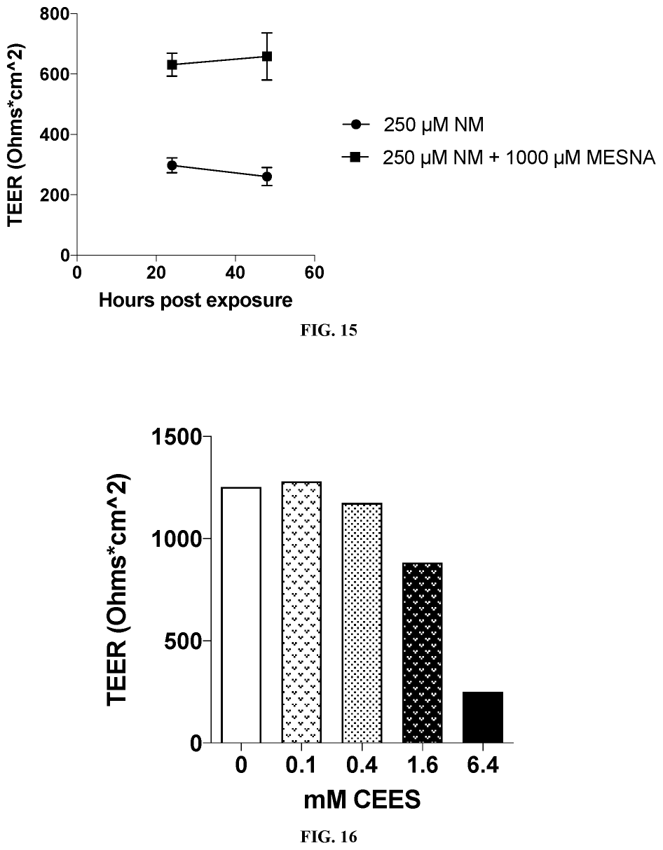

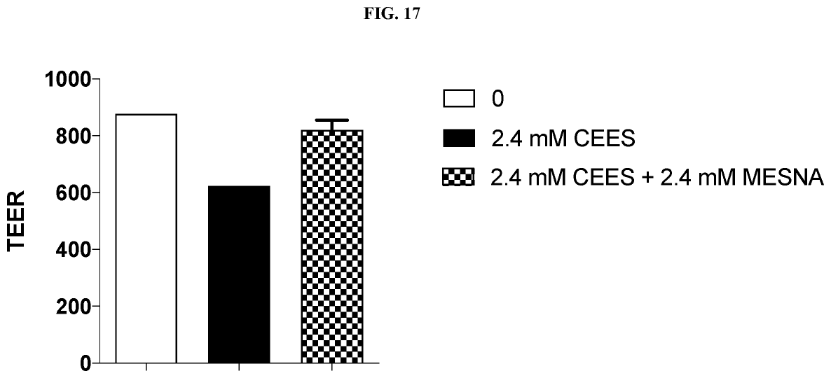

Nitrogen mustard (NM), like SM, is a multi-functional DNA crosslinking alkylating agent that has not been widely weaponized. NM was substituted for SM in some studies, with qualitatively similar results. For example, it was found that mixing 1 mM MESNA with 1 mM SM, immediately before addition to the apical surface, largely inhibited the TEER decrease due to SM. Conducting comparable studies with NM, it was found that control cells had TEER of 1,000 ohms*cm², maintaining this resistance for 5 days. NM at 62.5 uM caused almost no decline in TEER. By contrast, 250 uM NM decreased TEER by 50% (to 500 ohms*cm²), where it remained for 5 days (FIG. 14). Adding 1 mM MESNA to 250 uM NM, immediately before apical exposure to cells, prevented the decline in TEER, which remained steady 48 h (FIG. 15). Thus, NM, like SM, caused decline in TEER preventable by early addition of MESNA.

### Example 4: Effects of CEES on TEER and Reversal by MESNA

As with SM, similar studies were done with CEES (2-chloro-ethyl-ethylsulfide), an alkylating, monofunctional, non-crosslinking sulfur mustard. (CEES has only one terminal chlorine, while SM, and the NM used by us, have two.). Using the range of 0.1-6.4 mM CEES, a 6-fold decline in TEER was seen at 6.4 mM, (FIG. 16) similar to SM and NM; adding MESNA to CEES (2.4 mM for each) completely inhibited the CEES-dependent TEER decline. Thus, cell dysfunction due to 3 commonly studied mustards could be prevented by MESNA.

Additional evidence for the mechanism of MESNA's action was sought and it was suspected that the reactive group on MESNA is the sulfhydryl (—SH), and that MESNA's reaction with mustards might be shown by the presence or absence of the sulfhydryl.

### Example 5: Use of DTNB to Monitor MESNA's Reaction with Mustards

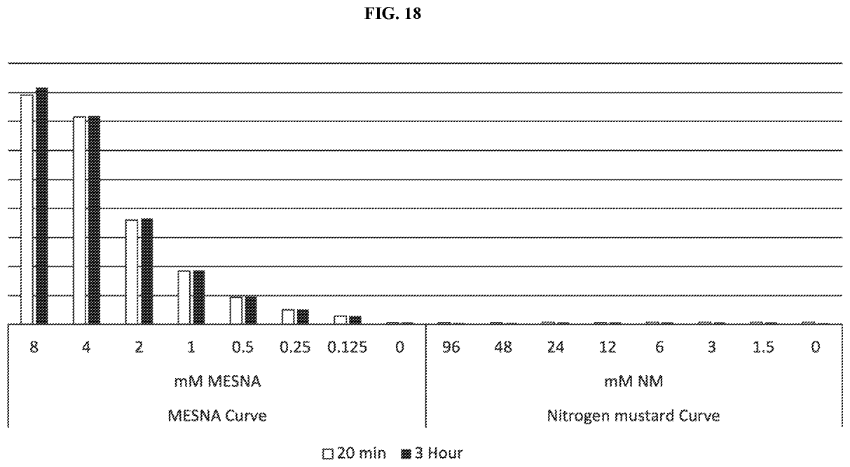

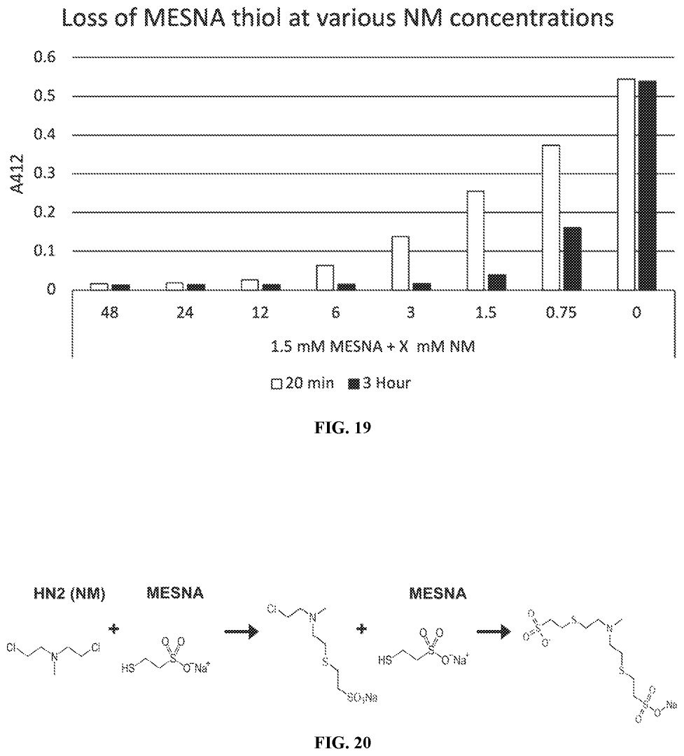

Ellman reagent (DTNB) was used to detect the free sulfhydryl group on MESNA. MESNA (0-8 mM) or NM (0-96 mM) was reacted with DTNB in buffer. MESNA caused an absorbance increase of Ellman reagent (412 nm) over that range, and that absorbance was stable for >3 hr (FIG. 18, left panel). By contrast, addition of NM without MESNA caused no increase in absorbance (FIG. 18, right panel). Next, holding [MESNA] constant at 1.5 mM, an increasing amount of NM was added and the reactions were incubated for 20 mins or 3 h. DTNB was added at the end of the reaction. In presence of NM, there was a decline in absorbance (412 nm) over time (FIG. 19). This suggests that a complex between MESNA and NM could be formed. At 8-fold NM excess (12:1.5 NM/MESNA), MESNA's thiol was rapidly depleted; at 2-fold NM excess (3:1.5 NM:MESNA), MESNA was depleted over time; and at equimolar NM and MESNA (1.5:1.5) there was still some reactive thiol at 3 h (FIG. 19). In each pair, left column is absorbance at 20 min, and right column is that for 3 h). Together, these results indicate that MESNA may act as ‘scavenger’ for mustards SM, NM, and CEES. Such interaction could decrease SM toxicity in vivo.

### Example 6

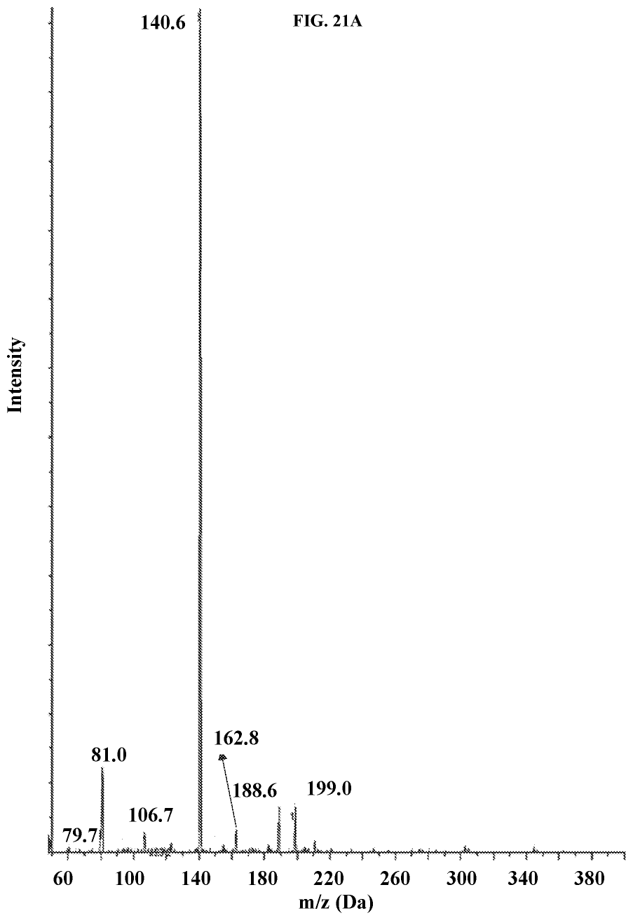

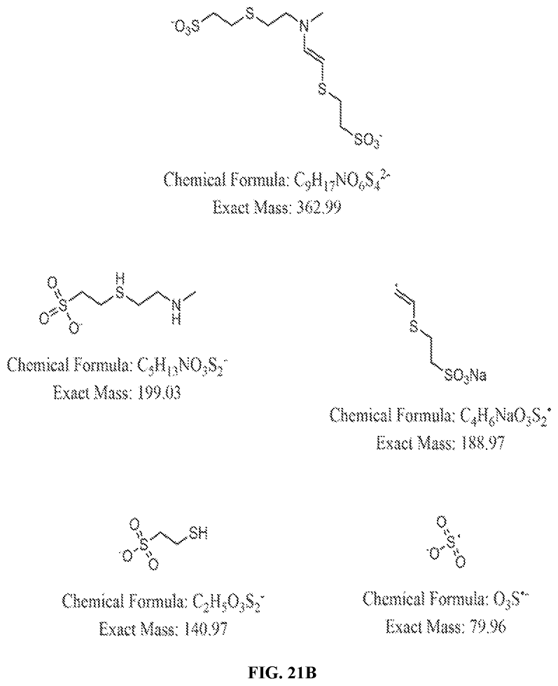

Using mass spectrometry, it was found that the reaction between MESNA and HN2 (NM) likely proceeds from a 1:1 MESNA:NM reaction to a 2:1 MESNA:NM product (the larger, 362.9 Da molecule shown FIG. 20). The mass spectrum of the likely final MESNA:NM product is shown in FIG. 21A, with likely structures of the abundant fragments illustrated in FIG. 21B.

### Enumerated Embodiments

The following enumerated embodiments are provided, the numbering of which is not to be construed as designating levels of importance.

Embodiment 1 provides a method of treating a subject exposed to a toxic inhaled chemical, the method comprising administering to the subject a therapeutically effective amount of 2-mercaptoethane sulfonic acid, or a salt or solvate thereof.

Embodiment 2 provides the method of embodiment 1, wherein the 2-mercaptoethane sulfonic acid salt is sodium 2-mercaptoethane sulfonate or a salt thereof.

Embodiment 3 provides the method of embodiments 1-2, wherein the toxic inhaled chemical is sulfur mustard, chlorine, methyl mercaptan, nitrogen mustard, 2-chloro-ethyl-ethylsulfide (CEES), methyl isocyanate, or a combination thereof.

Embodiment 4 provides the method of embodiments 1-3, wherein 2-mercaptoethane sulfonic acid, or salt or solvate thereof, is administered to the subject via one of the following routes: oral, intravenous, subcutaneous, intra-osseus, intramuscular, intraperitoneal, intratracheal, cutaneous, or intra-ocular.

Embodiment 5 provides the method of embodiments 1-4, wherein between about 120 mg/m² to about 720 mg/m² of 2-mercaptoethane sulfonic acid, or a salt or solvate thereof, is administered to the subject.

Embodiment 6 provides the method of embodiments 1-5, wherein about 120 mg/m² to about 360 mg/m² of 2-mercaptoethane sulfonic acid, or a salt or solvate thereof, is administered intravenously to the subject.

Embodiment 7 provides the method of embodiments 1-5, wherein about 240 mg/m² to about 720 mg/m² of 2-mercaptoethane sulfonic acid, or a salt or solvate thereof, is administered orally to the subject.

Embodiment 8 provides the method of embodiments 1-7, wherein 2-mercaptoethane sulfonic acid, or a salt or solvate thereof, is administered to the subject immediately to about ten hours after the subject was exposed to the toxic inhaled chemical.

Embodiment 9 provides the method of embodiments 1-8, wherein 2-mercaptoethane sulfonic acid, or a salt or solvate thereof, is administered to the subject within about 20 minutes after the subject was exposed to the toxic inhaled chemical.

Embodiment 10 provides the method of embodiments 1-8, wherein 2-mercaptoethane sulfonic acid, or a salt or solvate thereof, is administered to the subject about two hours to about eight hours after the subject was exposed to the toxic inhaled chemical.

Embodiment 11 provides the method of any of the preceding embodiments, wherein:
(i) a first dose of 2-mercaptoethane sulfonic acid, or a salt or solvate thereof, is administered intravenously to the subject within about 20 minutes after the subject was exposed to the toxic inhaled chemical;
(ii) a second dose of 2-mercaptoethane sulfonic acid, or a salt or solvate thereof, is administered intravenously to the subject about 4 hours after the subject was exposed to the toxic inhaled chemical; and
(iii) a third dose of 2-mercaptoethane sulfonic acid, or a salt or solvate thereof, is administered intravenously to the subject about 8 hours after the subject was exposed to the toxic inhaled chemical.

Embodiment 12 provides the method of any of the preceding embodiments, wherein:
(i) a first dose of 2-mercaptoethane sulfonic acid, or a salt or solvate thereof, is administered intravenously to the subject within about 20 minutes after the subject was exposed to the toxic inhaled chemical;
(ii) a second dose of 2-mercaptoethane sulfonic acid, or a salt or solvate thereof, is administered orally to the subject about 2 hours after the subject was exposed to the toxic inhaled chemical; and
(iii) a third dose of 2-mercaptoethane sulfonic acid, or a salt or solvate thereof, is administered orally to the subject about 6 hours after the subject was exposed to the toxic inhaled chemical.

Embodiment 13 provides the method of embodiments 1-12, further comprising administering to the subject a therapeutically effective amount of a fibrinolytic agent.

Embodiment 14 provides the method of embodiments 1-13, wherein the fibrinolytic agent is tissue plasminogen activator (tPA) or an analog thereof.

Embodiment 15 provides the method of embodiments 1-14, wherein between about 0.4 mg/kg to about 1.0 mg/kg tPA or the analog thereof is administered to the subject.

Embodiment 16 provides the method of embodiments 1-15, wherein tPA or the analog thereof is administered to the subject about six hours to about 24 hours after the subject was exposed to the toxic inhaled chemical.

Embodiment 17 provides the method of embodiments 1-16, wherein tPA or the analog thereof is administered to the subject when the subject shows at least one of symptoms (i)-(v):
(i) acutely worsening respiratory distress;
(ii) acutely worsening hypoxemia;
(iii) evidence of acutely worsening upper and/or lower airways obstruction selected from stridor, suprasternal retractions, wheezing, chest retractions, and combinations thereof;
(iv) fibrinous casts, clots, or pseudomembranes; or
(v) a blood-oxygen saturation≤85%.

Embodiment 18 provides the method of embodiments 1-17, wherein tPA or the analog thereof is administered to the subject via a bronchoscopy or an endotracheal tube.

Embodiment 19 provides the method of embodiments 1-18, wherein the method improves survivability of the subject, peripheral arterial oxygen saturation in the subject, or a combination thereof.

Embodiment 20 provides the method of embodiments 1-19, wherein the method prevents or decreases airway coagulation, cast formation, or a combination thereof in the subject.

Embodiment 21 provides the method of embodiments 1-20, wherein the subject is a mammal.

Embodiment 22 provides the method of embodiments 1-21, wherein the subject is a human subject.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating a subject exposed to a toxic inhaled chemical, the method comprising administering to the subject a therapeutically effective amount of 2-mercaptoethane sulfonic acid, or a salt or solvate thereof.

2. The method of claim 1, wherein the 2-mercaptoethane sulfonic acid salt is sodium 2-mercaptoethane sulfonate, or a solvate thereof.

3. The method of claim 1, wherein the toxic inhaled chemical is sulfur mustard, chlorine gas, methyl mercaptan, nitrogen mustard, 2-chloro-ethyl-ethylsulfide (CEES), methyl isocyanate, or a combination thereof.

4. The method of claim 1, wherein 2-mercaptoethane sulfonic acid, or salt or solvate thereof, is administered to the subject via one of the following routes: oral, intravenous, subcutaneous, intra-osseus, intramuscular, intraperitoneal, intratracheal, cutaneous, or intra-ocular.

5. The method of claim 1, wherein between about 120 mg/m² to about 720 mg/m² of 2-mercaptoethane sulfonic acid, or a salt or solvate thereof, is administered to the subject.

6. The method of claim 1, wherein about 120 mg/m² to about 360 mg/m² of 2-mercaptoethane sulfonic acid, or a salt or solvate thereof, is administered intravenously to the subject.

7. The method of claim 1, wherein about 240 mg/m² to about 720 mg/m² of 2-mercaptoethane sulfonic acid, or a salt or solvate thereof, is administered orally to the subject.

8. The method of claim 1, wherein 2-mercaptoethane sulfonic acid, or a salt or solvate thereof, is administered to the subject immediately to about ten hours after the subject was exposed to the toxic inhaled chemical.

9. The method of claim 1, wherein 2-mercaptoethane sulfonic acid, or a salt or solvate thereof, is administered to the subject within about 20 minutes after the subject was exposed to the toxic inhaled chemical.

10. The method of claim 1, wherein 2-mercaptoethane sulfonic acid, or a salt or solvate thereof, is administered to the subject about two hours to about eight hours after the subject was exposed to the toxic inhaled chemical.

11. The method of claim 1, wherein:
(i) a first dose of 2-mercaptoethane sulfonic acid, or a salt or solvate thereof, is administered intravenously to the subject within about 20 minutes after the subject was exposed to the toxic inhaled chemical;
(ii) a second dose of 2-mercaptoethane sulfonic acid, or a salt or solvate thereof, is administered intravenously to the subject about 4 hours after the subject was exposed to the toxic inhaled chemical; and
(iii) a third dose of 2-mercaptoethane sulfonic acid, or a salt or solvate thereof, is administered intravenously to the subject about 8 hours after the subject was exposed to the toxic inhaled chemical.

12. The method of claim 1, wherein:
(i) a first dose of 2-mercaptoethane sulfonic acid, or a salt or solvate thereof, is administered intravenously to the subject within about 20 minutes after the subject was exposed to the toxic inhaled chemical;
(ii) a second dose of 2-mercaptoethane sulfonic acid, or a salt or solvate thereof, is administered orally to the subject about 4 hours after the subject was exposed to the toxic inhaled chemical; and
(iii) a third dose of 2-mercaptoethane sulfonic acid, or a salt or solvate thereof, is administered orally to the subject about 8 hours after the subject was exposed to the toxic inhaled chemical.

13. The method of claim 1, further comprising administering to the subject a therapeutically effective amount of a fibrinolytic agent.

14. The method of claim 13, wherein the fibrinolytic agent is tissue plasminogen activator (tPA) or an analog thereof.

15. The method of claim 14, wherein between about 0.4 mg/kg to about 1.0 mg/kg tPA or the analog thereof is administered to the subject.

16. The method of claim 14, wherein tPA or the analog thereof is administered to the subject about six hours to about 24 hours after the subject was exposed to the toxic inhaled chemical.

17. The method of claim 16, wherein tPA or the analog thereof is administered to the subject when the subject shows at least one of symptoms (i)-(v):
(i) acutely worsening respiratory distress;
(ii) acutely worsening hypoxemia;
(iii) evidence of acutely worsening upper and/or lower airways obstruction selected from stridor, suprasternal retractions, wheezing, chest retractions, and combinations thereof;
(iv) fibrinous casts, clots, or pseudomembranes; or
(v) a blood-oxygen saturation≤85%.

18. The method of claim 14, wherein tPA or the analog thereof is administered to the subject via a bronchoscopy or an endotracheal tube.

19. The method of claim 1, wherein the method improves survivability of the subject, peripheral arterial oxygen saturation in the subject, or a combination thereof.

20. The method of claim 1, wherein the method prevents or decreases airway coagulation, cast formation, or a combination thereof in the subject.

21. The method of claim 1, wherein the subject is a mammal.

22. The method of claim 1, wherein the subject is a human.
